(19) United States
(12) Patent Application Publication
(10) Pub. No.: US 2023/0260605 A1
(43) Pub. Date: Aug. 17, 2023

(54) COGNITIVE ENHANCEMENT TRAINING METHOD AND SYSTEM BASED ON NEURAL REGULATION

(71) Applicant: BEIJING WISPIRIT TECHNOLOGY CO., LTD, Beijing (CN)

(72) Inventors: Sha LI, Beijing (CN); Zhujiang MA, Beijing (CN); Xiaoyi WANG, Beijing (CN)

(73) Assignee: BEIJING WISPIRIT TECHNOLOGY CO., LTD, Beijing, BJ (CN)

(21) Appl. No.: 18/148,819

(22) Filed: Dec. 30, 2022

Related U.S. Application Data
(63) Continuation of application No. PCT/CN2022/121094, filed on Sep. 23, 2022.

(30) Foreign Application Priority Data
Feb. 17, 2022 (CN) 202210148357.9

(51) Int. Cl. G16H 10/20; G16H 50/20

(52) U.S. Cl. CPC G16H 10/20; G16H 50/20

(57) ABSTRACT

Disclosed are a cognitive enhancement training method and system based on neural regulation. The method includes the following steps: obtaining a cognitive enhancement need of a user; obtaining a rhythm frequency based on the cognitive enhancement need of the user; obtaining a sensory stimulation mode for the user to perform human-computer interaction; obtaining a human-computer interaction scheme based on the cognitive enhancement need of the user; pushing the human-computer interaction scheme to the user to perform human-computer interaction training at the rhythm frequency and in the sensory stimulation mode; obtaining a human-computer interaction result of the user, and evaluating the human-computer interaction result; and adjusting the human-computer interaction scheme according to the human-computer interaction scheme and a human-computer interaction evaluation result, and pushing the adjusted human-computer interaction scheme to the user to perform a next human-computer interaction training until the purpose of cognitive enhancement of the user is achieved.

### BACKGROUND

### Technical Field

The present disclosure relates to a cognitive enhancement training method based on neural regulation, and also relates to a corresponding cognitive enhancement training system, belonging to the field of medical care informatics.

### Related Art

A neural regulation technology is a research and training means widely used in the field of neuroscience at present. Combined with neural information detection methods, the neural regulation technology is of great significance to the research on neural circuit connections, animal behaviors, nervous system mechanisms and nervous system pathogenesis.

Relevant researches show that the cognition of both music rhythm and motion rhythm is synchronous, that is, a patient is stimulated by an auditory rhythm to activate the motor cortex of the brain so as to cause the motor cortex to carry out motion planning, and finally is innervated by the spinal cord motor neurons to cause muscle contraction, thereby generating the motion rhythm. Therefore, in the field of cognition, the nervous system can be trained by regular stimulation (that is, rhythms) such as sound, light and electricity.

Different senses have different perceptions to rhythm. In general, tactility is the most sensitive, followed by audition, and vision comes last. However, regardless of the sensory channel, some mental users, such as attention deficit hyperactivity disorder, autism spectrum disorder, bipolar disorder, schizophrenia and stroke, or other normal people are lacking and uncoordinated in perception of rhythms. Therefore, training of rhythm not only helps users establish their own rhythm and restore relevant cognitive functions, but also helps users restore their social functions.

At present, most of the clinical researches have discussed the evaluation of rhythm-related functions, and most of them focus on the impact of music rhythm changes on users. The research scope is too narrow, such as focusing on the evaluation and treatment of muscle rhythm, and the relationship between rhythm treatment and cognitive development is rarely considered. Furthermore, the current research on rhythm is mainly in the measurement and evaluation stage, which still fails to achieve the purpose of cognitive enhancement of users by rhythm-related tasks.

### SUMMARY

A primary technical problem to be solved by the present disclosure is to provide a cognitive enhancement training method based on neural regulation.

Another technical problem to be solved by the present disclosure is to provide a cognitive enhancement training system based on neural regulation.

To achieve the above objective, the present disclosure adopts the following technical solutions:

According to a first aspect of an embodiment of the present disclosure, a cognitive enhancement training method based on neural regulation is provided, which includes the following steps:

obtaining a rhythm frequency for a user to perform human-computer interaction;

obtaining a sensory stimulation mode for the user to perform human-computer interaction;

obtaining a human-computer interaction scheme for the user to perform human-computer interaction;

pushing the human-computer interaction scheme to the user to perform human-computer interaction at the rhythm frequency and in the sensory stimulation mode;

obtaining a human-computer interaction result of the user, and evaluating the human-computer interaction result; and

adjusting the human-computer interaction scheme according to the human-computer interaction scheme and a human-computer interaction evaluation result of the user, and pushing the adjusted human-computer interaction scheme to the user to perform a next human-computer interaction training until the purpose of cognitive enhancement of the user is achieved.

Preferably, the obtaining a rhythm frequency for a user to perform human-computer interaction specifically includes:

if the user is diagnosed with cognitive diseases, according to a preset correspondence between the diseases and rhythm frequencies, based on a certain disease in the user diseases, obtaining a rhythm frequency corresponding to the certain disease; and

if the user is not diagnosed with cognitive diseases, according to an ability to be enhanced of the user, obtaining a rhythm frequency corresponding to the ability to be enhanced of the user.

Preferably, the sensory stimulation mode includes: at least a visual mode, an auditory mode, a tactile mode, a visual-auditory mode, a visual-tactile mode, an auditory-tactile mode and a visual-auditory-tactile mode.

Preferably, the obtaining a human-computer interaction scheme for the user to perform human-computer interaction specifically includes:

if the user is diagnosed with cognitive diseases, according to a preset correspondence between the diseases and human-computer interaction schemes, based on a certain disease in the user diseases, obtaining a human-computer interaction scheme corresponding to the certain disease; and

if the user is not diagnosed with cognitive diseases, determining task types of human-computer interaction tasks based on an ability to be enhanced of the user, determining task levels of the human-computer interaction tasks based on a degree of the ability to be enhanced of the user, determining the task number of the human-computer interaction tasks based on an acceptable human-computer interaction intensity of the user, and obtaining a human-computer interaction scheme according to the task types, the task levels and the task number of the human-computer interaction tasks.

Preferably, if the user is not diagnosed with cognitive diseases, performing cognitive evaluation on the user, and based on a cognitive evaluation result of the user, obtaining the ability to be enhanced of the user, the degree of the ability to be enhanced and the acceptable human-computer interaction intensity.

Preferably, the task types of the human-computer interaction tasks include a rhythm perception type, a rhythm memory type and a rhythm learning type in order from low level to high level.

Preferably, the human-computer interaction tasks of the rhythm perception type include: at least a rhythm task with beats, a stress perception task, a difference and similarity rhythm discrimination task, and a wrong rhythm recognition task;

the human-computer interaction tasks of the rhythm memory type include: at least a rhythm task with interval beats, a rhythm imitation task, and a rhythm memory comparison task; and

the human-computer interaction tasks of the rhythm learning type include: at least a rhythm reasoning task, a rhythm playing task, and a rhythm creating task.

Preferably, after the obtaining a human-computer interaction scheme for the user to perform human-computer interaction, the method further includes:

according to personal factors of the user, under the human-computer interaction tasks of the same type, selecting a specific sub-task to adjust the human-computer interaction scheme;

where the personal factors of the user include: at least one or a combination of more of age, gender, personality and physical defects.

Preferably, the adjusting the human-computer interaction scheme according to the human-computer interaction scheme and a human-computer interaction evaluation result of the user, and pushing the adjusted human-computer interaction scheme to the user to perform a next human-computer interaction training until the purpose of cognitive enhancement of the user is achieved specifically includes:

in the previous three human-computer interaction schemes, increasing the task level of the next human-computer interaction scheme by one level based on the task level of the current human-computer interaction scheme; and

at the beginning of the fourth human-computer interaction scheme, if the evaluation result of the current human-computer interaction scheme is higher than the evaluation result of the previous human-computer interaction scheme, increasing the task level of the next human-computer interaction scheme by one level, and if the evaluation result of the current human-computer interaction scheme is lower than the evaluation result of the previous human-computer interaction scheme, reducing the task level of the next human-computer interaction scheme by one level.

According to a second aspect of an embodiment of the present disclosure, a cognitive enhancement training system based on neural regulation is provided, which includes:

a data collection unit, connected with a central processing unit to obtain basic information of a user;

a rhythm frequency obtaining unit, connected with the central processing unit to obtain a rhythm frequency for the user to perform human-computer interaction;

a sensory stimulation mode obtaining unit, connected with the central processing unit to obtain a sensory stimulation mode for the user to perform human-computer interaction;

a human-computer interaction unit, connected with the central processing unit to obtain a human-computer interaction scheme and perform human-computer interaction with the user;

a human-computer interaction scheme evaluation unit, connected with the central processing unit to evaluate a human-computer interaction result of the user; and

a human-computer interaction scheme optimization unit, connected with the central processing unit and the human-computer interaction unit to adjust the human-computer interaction scheme based on an evaluation result of the human-computer interaction, and push the adjusted human-computer interaction scheme to the human-computer interaction unit;

where the central processing unit is configured to perform the above-mentioned cognitive enhancement training method.

Compared with the prior art, the cognitive enhancement training method and system provided by the present disclosure can achieve the purpose of enhancing the cognitive ability of users by setting different sensory stimulation modes and rhythm frequencies for users based on different purposes and needs of users. Furthermore, after a user completes a human-computer interaction scheme, a human-computer interaction result of the user is analyzed to evaluate the improvement effects on multiple cognitive functions of the user, such as perception, memory and social functions, and finally, a human-computer interaction evaluation result is obtained. Based on the evaluation result, the human-computer interaction scheme is adjusted by a computer-adaptive method, so as to form a next human-computer interaction scheme which is pushed to the user to perform next human-computer interaction training until the cognitive ability to be enhanced of the user reaches the normal level. Therefore, for different users and people with different needs for brain ability enhancement, the purpose of improvement can be achieved only by selecting an appropriate rhythm frequency and an appropriate sensory stimulation mode, and the contents are rich and numerous, thereby adapting to different human-computer interaction needs.

### BRIEF DESCRIPTION OF THE DRAWINGS

FIG. 1 is a schematic structural diagram of a cognitive enhancement training system based on neural regulation provided by an embodiment of the present disclosure.

FIG. 2 is a flowchart of a cognitive enhancement training method based on neural regulation provided by an embodiment of the present disclosure.

### DETAILED DESCRIPTION

The technical content of the present disclosure is described in detail below with reference to the accompanying drawings and specific embodiments.

In the field of cognition, neural regulation can stimulate patients according to preset rules (that is, rhythms) through acoustics, optics, perception and other aspects, so as to achieve the purpose of relieving diseases. Music rhythms, motion rhythms and optical rhythms are collectively referred to as rhythms. The rhythms are taken as an example for description below.

In an embodiment of the present disclosure, first, a rhythm frequency mapping library is pre-established according to the existing literature research and a large number of experimental data. In the rhythm frequency mapping library, one rhythm frequency corresponds to one disease, that is, human-computer interaction tasks at this rhythm frequency can effectively improve the symptoms of users with the corresponding diseases. For example, human-computer interaction tasks at a rhythm frequency of 40 HZ can effectively improve the symptoms of users with Alzheimer's disease, human-computer interaction tasks at a rhythm frequency of 8-13 HZ can effectively predict the degree of speech development of autistic children, and human-computer interaction tasks at a rhythm frequency of 6 HZ can help improve individual motor skills and prefrontal functions.

Furthermore, the present disclosure also pre-establishes a human-computer interaction scheme mapping library according to human-computer interaction results of a large number of users with different diseases. In the human-computer interaction scheme mapping library, each disease corresponds to a human-computer interaction scheme, and the human-computer interaction scheme includes at least the task types, task levels and task number of human-computer interaction tasks. It can be understood that the human-computer interaction scheme is a preferred human-computer interaction scheme obtained by analyzing the human-computer interaction result of the user with the same disease after human-computer interaction. When a user has the same disease, the human-computer interaction scheme can be directly pushed to the user. However, other human-computer interaction schemes can also be pushed to the user according to the cognitive enhancement need of the user.

In addition, in the embodiments of the present disclosure, it can be understood that each human-computer interaction scheme can only help improve a certain disease of the user. For example, when a user suffers from all of Alzheimer's disease, autism and stroke, each human-computer interaction scheme can only help improve one of the diseases, and if another disease needs to be improved, another different human-computer interaction scheme is required.

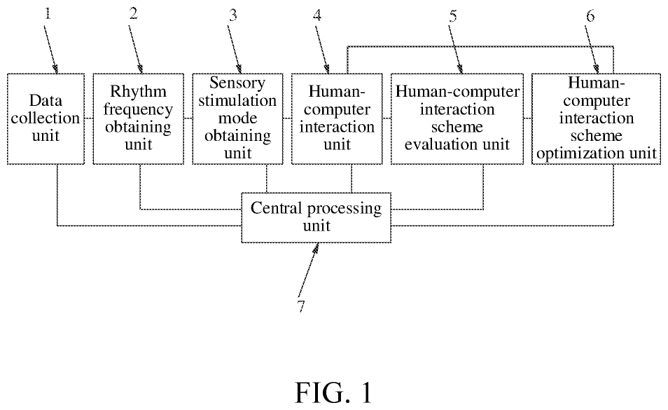

FIG. 1 shows a cognitive enhancement training system based on neural regulation provided by an embodiment of the present disclosure, including: at least a data collection unit 1, a rhythm frequency obtaining unit 2, a sensory stimulation mode obtaining unit 3, a human-computer interaction unit 4, a human-computer interaction scheme evaluation unit 5, a human-computer interaction scheme optimization unit 6, and a central processing unit 7.

The data collection unit 1 is connected with the central processing unit 7 to collect basic information of a user, such as name, gender, age and diseases. An initial training scheme is provided according to the basic information of a patient. The rhythm frequency obtaining unit 2 is connected with the central processing unit 7 to obtain a rhythm frequency for the user to perform human-computer interaction. The sensory stimulation mode obtaining unit 3 is connected with the central processing unit 7 to obtain a sensory stimulation mode for the user to perform human-computer interaction. The human-computer interaction unit 4 is connected with the central processing unit 7 to obtain a human-computer interaction scheme and perform human-computer interaction with the user. The human-computer interaction scheme evaluation unit 5 is connected with the central processing unit 7 to evaluate a human-computer interaction result of the user. The human-computer interaction scheme optimization unit 6 is connected with the central processing unit 7 and the human-computer interaction unit 4 to adjust the human-computer interaction scheme based on an evaluation result of the human-computer interaction, and push the adjusted human-computer interaction scheme to the human-computer interaction unit 4. The central processing unit 7 is configured to perform a cognitive enhancement training method based on neural regulation.

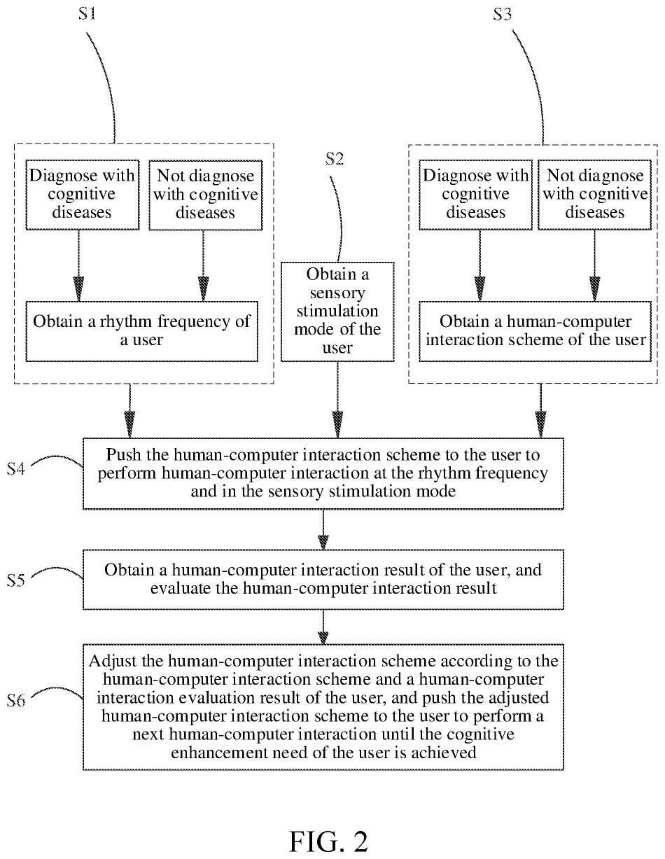

A cognitive enhancement training method based on neural regulation provided by an embodiment of the present disclosure is described in detail below with reference to FIG. 2. In an embodiment of the present disclosure, the cognitive enhancement training method includes at least the following steps:

S1: A rhythm frequency for a user to perform human-computer interaction is obtained.

Specifically, in this embodiment of the present disclosure, a rhythm frequency may be obtained by two ways, namely by means of diseases or the cognitive ability to be enhanced of the user.

The two obtaining ways are described in detail below:

The first obtaining way: if the user is diagnosed with cognitive diseases, after the data collection unit 1 obtains the disease information of the user, by using a pre-established rhythm frequency mapping library, for a certain disease of the user, a rhythm frequency corresponding to the disease can be obtained directly by the rhythm frequency obtaining unit 2. For example: if the user disease obtained by the data collection unit 1 is Alzheimer's disease, the rhythm frequency obtained by the rhythm frequency obtaining unit 2 is 40 HZ.

The second obtaining way: if the user is not diagnosed with cognitive diseases, it is necessary to evaluate the basic cognitive ability of the user by cognitive assessment (such as scale or cognitive paradigm), so as to determine the cognitive impairment of the user according to a cognitive evaluation result. Then, the cognitive ability to be enhanced of the user, the degree of the ability to be enhanced and the acceptable human-computer interaction intensity are obtained by the data collection unit 1. Specifically, in this embodiment of the present disclosure, for a certain cognitive ability to be enhanced of the user, there may be one or more corresponding rhythm frequencies. If there is a rhythm frequency corresponding to the cognitive ability, the rhythm frequency is obtained directly. If there are a plurality of rhythm frequencies corresponding to the cognitive ability, the rhythm frequency selected by the user is obtained.

S2: A sensory stimulation mode for the user to perform human-computer interaction is obtained.

Specifically, in this embodiment of the present disclosure, the sensory stimulation mode may include seven modes: a visual mode, an auditory mode, a tactile mode, a visual-auditory mode, a visual-tactile mode, an auditory-tactile mode and a visual-auditory-tactile mode. The visual mode, the auditory mode and the tactile mode belong to a single sensory stimulation mode, and the visual-auditory mode, the visual-tactile mode, the auditory-tactile mode and the visual-auditory-tactile mode belong to a comprehensive sensory stimulation mode. Of course, in other embodiments, the sensory stimulation mode may further include a single olfactory mode, a comprehensive sensory stimulation mode formed by combining olfaction with tactility, audition and vision, etc. The forms of rhythm cognitive training in the visual mode and the auditory mode are diverse. The rhythm training in the visual mode includes but is not limited to the dynamic presentation of point or line or picture like pictures, or the static presentation according to a certain regularity (such as three long and one short and similar forms), or may be some physiological rhythms (such as circadian rhythms, heartbeats, electroencephalogram and breathing fluctuation mode), or may be the swinging, swimming, running, etc. of animals. The rhythms in the auditory mode may be ordinary music rhythms, regular whops, physiological fluctuation sound, etc., which may be trained in the form of binaural synchronous presentation or binaural asynchronous presentation. In other words, the visual mode and auditory mode of rhythms and the ordinary cognitive training have cross fields.

It can be understood that different sensory stimulation modes have different human-computer interaction effects. According to sensitivity, the tactility is the most sensitive, followed by audition, and finally vision. In general, the human-computer interaction effect in the comprehensive sensory stimulation mode is higher than the human-computer interaction effect in the single sensory stimulation mode. Therefore, in this embodiment of the present disclosure, the default sensory stimulation mode is the visual-auditory-tactile mode. In addition, the user can set different sensory stimulation modes according to different purposes.

If the user sets a sensory stimulation mode, the sensory stimulation mode set by the user is obtained by the sensory stimulation mode obtaining unit 3. If the user does not set a sensory stimulation mode, the system default visual-auditory-tactile mode is obtained by the sensory stimulation mode obtaining unit 3.

S3: A human-computer interaction scheme for the user to perform human-computer interaction is obtained.

Specifically, the human-computer interaction scheme includes at least the task types, task levels and task number of human-computer interaction tasks. In this embodiment of the present disclosure, a human-computer interaction scheme may be obtained by two ways, namely by means of disease information of the user or needs of the user. The following describes how to obtain a human-computer interaction scheme by two ways in detail:

The first obtaining way: if the user is diagnosed with cognitive diseases, after the data collection unit 1 obtains the disease information of the user, by using a pre-established human-computer interaction scheme mapping library, for a certain disease of the user, a human-computer interaction scheme corresponding to the disease can be obtained by the human-computer interaction unit 4. For example, the first human-computer interaction scheme corresponds to Alzheimer's disease, the second human-computer interaction scheme corresponds to autism, the third human-computer interaction scheme corresponds to stroke, etc. If the user disease obtained by the data collection unit 1 is Alzheimer's disease, the first human-computer interaction scheme is obtained by the human-computer interaction unit 4. If the user disease obtained by the data collection unit 1 is autism, the second human-computer interaction scheme is obtained by the human-computer interaction unit 4.

Furthermore, it can be understood that the human-computer interaction schemes preset in the human-computer interaction scheme mapping library are only preferred schemes obtained through a large number of experiments. If the user has special needs, even if a certain disease of the user is obtained by the data collection unit 1, the human-computer interaction scheme corresponding to the disease may not be selected, but the human-computer interaction scheme is adjusted according to the special needs of the user (for example: the level of the human-computer interaction task is increased or reduced, the number of the human-computer interaction tasks is increased or reduced, etc.). Moreover, the human-computer interaction scheme is a basic scheme corresponding to the disease and is irrelevant to the degree of the disease of the user, that is, the basic scheme remains unchanged regardless of the severity degree of the disease of the user, and the basic scheme is subsequently adjusted according to the human-computer interaction result of the user to adapt to different users. In this way, the human-computer interaction schemes preset in the human-computer interaction scheme mapping library can be prevented from being too miscellaneous, which may cause system load or user selection difficulty. Furthermore, the flexibility of the human-computer interaction scheme is considered, and a targeted human-computer interaction scheme can be recommended according to different situations of users.

The second obtaining way: if the user is not diagnosed with cognitive diseases, according to a cognitive evaluation result of the user, the ability to be enhanced of the user, the degree of the ability to be enhanced and the acceptable human-computer interaction intensity are obtained in order. Then, task types of human-computer interaction tasks are determined based on the ability to be enhanced of the user, task levels of the human-computer interaction tasks are determined based on the degree of the ability to be enhanced of the user, and the task number of the human-computer interaction tasks is determined based on the acceptable human-computer interaction intensity of the user. Finally, a human-computer interaction scheme is obtained by the human-computer interaction unit 4 based on the task types, task levels and task number of the human-computer interaction tasks.

For example, if the ability to be enhanced of the user is attention, the task type of the human-computer interaction task corresponding to the ability is determined as rhythm perception ability training; if the degree of the ability to be enhanced of the user is moderate, the level of the human-computer interaction task is determined as Level 3 (there are 5 levels in total: Level 1 corresponds to normal users, Level 2 corresponds to users with mild impairments, Level 3 corresponds to users with moderate impairments, Level 4 corresponds to users with severe impairments, and Level 5 corresponds to users with atypical impairments (a certain ability is extremely strong)); and if the user can accept no more than 5 human-computer interaction tasks, the number of the human-computer interaction tasks is determined as 3-5. Therefore, with reference to the three factors, a human-computer interaction scheme corresponding to the cognitive enhancement need of the user is determined comprehensively.

In this embodiment of the present disclosure, the task types of the human-computer interaction tasks include a rhythm perception type, a rhythm memory type and a rhythm learning type in order from low level to high level. The human-computer interaction tasks of the rhythm perception type include: at least a rhythm task with beats, a stress perception task, a difference and similarity rhythm discrimination task, and a wrong rhythm recognition task. The human-computer interaction tasks of the rhythm memory type include: at least a rhythm task with interval beats, a rhythm imitation task, and a rhythm memory comparison task. The human-computer interaction tasks of the rhythm learning type include: at least a rhythm reasoning task, a rhythm playing task, and a rhythm creating task. Preferably, the user can select task types of the human-computer interaction tasks from low to high in order to achieve the purpose of gradual progress. Of course, the user can also directly select rhythm learning tasks at the highest level, depending on the actual needs of the user.

It can be understood that in this embodiment of the present disclosure, after the task type of human-computer interaction tasks is determined based on the ability to be enhanced of the user, the human-computer interaction tasks of this task type can be randomly pushed to the user. For example, if it is determined that the cognitive enhancement of the user in the aspect of rhythm perception is needed after cognitive assessment, one of rhythm perception tasks is randomly pushed according to the needs of the user, specifically a specific number of tasks in the rhythm task with beats, the stress perception task, the difference and similarity rhythm discrimination task, and the wrong rhythm recognition task.

In another embodiment, regardless of whether the human-computer interaction scheme is obtained by the first way or the second way, the human-computer interaction scheme needs to be optimized with reference to the personal factors of the user (such as age, hobbies, personality, and physical defects). Specifically, if the human-computer interaction scheme is obtained by the first way, that is, a basic scheme is obtained according to a corresponding disease, based on the basic scheme, specific human-computer interaction tasks are adaptively adjusted according to the personal factors of the user. If the human-computer interaction scheme is obtained by the second way, after the task type of human-computer interaction tasks is determined based on the ability to be enhanced of the user, under this task type, specific tasks of the same task type are recommended with reference to the personal factors of the user.

For example, if a user is a patient with mild cognitive impairment (MCI), corresponding to tasks of the rhythm memory type, considering that the interest preferences of the user include listening to music, and the user is a 70-year-old female elderly, it is preferred to recommend auditory rhythm memory interaction tasks with high level difficulty in a human-computer interaction scheme for memory intervention.

In another example, if a user is a patient with severe cognitive impairment, corresponding to tasks of the rhythm memory type, considering that the interest preferences of the user include sports, and the user is a 56-year-old healthy male elderly without physical diseases, it is preferred to recommend tactile rhythm memory interaction tasks with primary difficulty.

In another example, if a user is a 6-year-old child with mild dyslexia, corresponding to tasks of the rhythm learning type, considering that the child has no morbid hearing impairment and has a good ability to distinguish syllables, it is preferred to recommend auditory rhythm learning interaction tasks with high level difficulty.

Taking the rhythm memory type as an example, the human-computer interaction tasks of the rhythm memory type include three sub-tasks: a rhythm memory task with interval beats, a rhythm imitation task, and a rhythm memory comparison task. The task interaction duration is fixed. Each task has three difficulty levels: primary difficulty, intermediate difficulty, and advanced difficulty. Specific difficulty levels are combined and differentiated according to the number of beats in a single group of rhythms (1 beat, 2 beats, 3 beats) and time intervals (0.5 s, 1 s, 1.5 s) as indexes. For example, the primary difficulty is determined when the number of beats in a single group is minimum and the time interval is longest, and the advanced difficulty is determined when there are many beats and the time interval is short.

As shown in Table 1, push rules between diseases and tasks are: the milder the disease is, the higher the task level is, and on the contrary, the lower the task level is.

TABLE 1
Push relationship between diseases and task levels

| Disease-task | Degree of disease | | |
|---|---|---|---|
| push level | Mild | Moderate | Severe |
| Task level | Advanced | Intermediate | Primary |

S4: The human-computer interaction scheme is pushed to the user to perform human-computer interaction training at the rhythm frequency and in the sensory stimulation mode.

After the rhythm frequency, the sensory stimulation mode and the human-computer interaction scheme are determined, the human-computer interaction unit 4 is configured to push the human-computer interaction scheme to the user to perform human-computer interaction training at the rhythm frequency and in the sensory stimulation mode. When the user performs human-computer interaction training, the human-computer interaction process of the user is recorded by the system for subsequent evaluation of a human-computer interaction result. Specifically, tactile perception can be realized by screen vibration, or tactile stimulation can be performed by a VR or electrode device.

S5: A human-computer interaction result of the user is obtained, and the human-computer interaction result is evaluated.

Specifically, the human-computer interaction result of the user is obtained by the human-computer interaction process recorded by the system. The human-computer interaction result includes: at least a task score for the user to perform human-computer interaction, a response time, an error rate, a score change, etc. The human-computer interaction result is analyzed by the human-computer interaction scheme evaluation unit 5 to evaluate the improvement effects on multiple cognitive functions of the user, such as perception, memory and social functions, and thus, a human-computer interaction evaluation result of the user is obtained.

S6: The human-computer interaction scheme is adjusted according to the human-computer interaction scheme and the human-computer interaction evaluation result of the user, and the adjusted human-computer interaction scheme is pushed to the user to perform the next human-computer interaction training until the purpose of cognitive enhancement of the user is achieved.

Specifically, in the previous three human-computer interaction schemes, the task level of the next human-computer interaction scheme is increased by one level based on the task level of the current human-computer interaction scheme.

At the beginning of the fourth human-computer interaction scheme, if the evaluation result of the current human-computer interaction scheme is higher than the evaluation result of the previous human-computer interaction scheme, the task level of the next human-computer interaction scheme is increased by one level, and if the evaluation result of the current human-computer interaction scheme is lower than the evaluation result of the previous human-computer interaction scheme, the task level of the next human-computer interaction scheme is reduced by one level.

In the process of cognitive enhancement of the user, the human-computer interaction scheme is continuously improved and adjusted in real time according to the cognitive enhancement situation of the user, thereby helping the user better enhance the cognitive ability until the purpose of cognitive enhancement of the user is achieved.

In conclusion, the cognitive enhancement training method and system based on neural regulation provided by the present disclosure can achieve the purpose of enhancing the cognitive ability of users by setting different sensory stimulation modes and rhythm frequencies for users based on different purposes and needs of users. Furthermore, after a user completes a human-computer interaction scheme, a human-computer interaction result of the user is analyzed to evaluate the improvement effects on multiple cognitive functions of the user, such as perception, memory and social functions, and finally, a human-computer interaction evaluation result is obtained. Based on the evaluation result, the human-computer interaction scheme is adjusted by a computer-adaptive method, so as to form a next human-computer interaction scheme which is pushed to the user to perform next human-computer interaction training until the cognitive ability to be enhanced of the user reaches the normal level. Therefore, for different users and people with different needs for brain ability enhancement, the purpose of improvement can be achieved only by selecting an appropriate rhythm frequency and an appropriate sensory stimulation mode, and the contents are rich and numerous, thereby adapting to different human-computer interaction needs.

The cognitive enhancement training method and system based on neural regulation provided by the present disclosure are described in detail above. For a person of ordinary skill in the art, any obvious modifications made to the present disclosure without departing from the essence of the present disclosure will constitute an infringement of patent rights of the present disclosure, and corresponding legal liabilities will be born.

What is claimed is:

1. A cognitive enhancement training method based on neural regulation, comprising the following steps:
obtaining a rhythm frequency for a user to perform human-computer interaction;
obtaining a sensory stimulation mode for the user to perform human-computer interaction;
obtaining a human-computer interaction scheme for the user to perform human-computer interaction;
pushing the human-computer interaction scheme to the user to perform human-computer interaction at the rhythm frequency and in the sensory stimulation mode;
obtaining a human-computer interaction result of the user, and evaluating the human-computer interaction result; and
adjusting the human-computer interaction scheme according to the human-computer interaction scheme and a human-computer interaction evaluation result of the user, and pushing the adjusted human-computer interaction scheme to the user to perform a next human-computer interaction training until the purpose of cognitive enhancement of the user is achieved.

2. The cognitive enhancement training method according to claim 1, wherein the obtaining a rhythm frequency for a user to perform human-computer interaction specifically comprises:
if the user is diagnosed with cognitive diseases, according to a preset correspondence between the diseases and rhythm frequencies, based on a certain disease in the user diseases, obtaining a rhythm frequency corresponding to the certain disease; and
if the user is not diagnosed with cognitive diseases, according to an ability to be enhanced of the user, obtaining a rhythm frequency corresponding to the ability to be enhanced of the user.

3. The cognitive enhancement training method according to claim 1, wherein the sensory stimulation mode comprises: at least a visual mode, an auditory mode, a tactile mode, a visual-auditory mode, a visual-tactile mode, an auditory-tactile mode and a visual-auditory-tactile mode.

4. The cognitive enhancement training method according to claim 1, wherein the obtaining a human-computer interaction scheme for the user to perform human-computer interaction specifically comprises:
if the user is diagnosed with cognitive diseases, according to a preset correspondence between the diseases and human-computer interaction schemes, based on a certain disease in the user diseases, obtaining a human-computer interaction scheme corresponding to the certain disease; and
if the user is not diagnosed with cognitive diseases, determining task types of human-computer interaction tasks based on an ability to be enhanced of the user, determining task levels of the human-computer interaction tasks based on a degree of the ability to be enhanced of the user, determining the task number of the human-computer interaction tasks based on an acceptable human-computer interaction intensity of the user, and obtaining a human-computer interaction scheme according to the task types, the task levels and the task number of the human-computer interaction tasks.

5. The cognitive enhancement training method according to claim 4, wherein
if the user is not diagnosed with cognitive diseases, performing cognitive evaluation on the user, and based on a cognitive evaluation result of the user, obtaining the ability to be enhanced of the user, the degree of the ability to be enhanced and the acceptable human-computer interaction intensity.

6. The cognitive enhancement training method according to claim 4, wherein
the task types of the human-computer interaction tasks comprise a rhythm perception type, a rhythm memory type and a rhythm learning type in order from low level to high level.

7. The cognitive enhancement training method according to claim 6, wherein
the human-computer interaction tasks of the rhythm perception type comprise: at least a rhythm task with beats, a stress perception task, a difference and similarity rhythm discrimination task, and a wrong rhythm recognition task;
the human-computer interaction tasks of the rhythm memory type comprise: at least a rhythm task with interval beats, a rhythm imitation task, and a rhythm memory comparison task; and
the human-computer interaction tasks of the rhythm learning type comprise: at least a rhythm reasoning task, a rhythm playing task, and a rhythm creating task.

8. The cognitive enhancement training method according to claim 4, wherein after the obtaining a human-computer interaction scheme for the user to perform human-computer interaction, the method further comprises:
according to personal factors of the user, under the human-computer interaction tasks of the same type, selecting a specific sub-task to adjust the human-computer interaction scheme;
wherein the personal factors of the user comprise: at least one or a combination of more of age, gender, personality and physical defects.

9. The cognitive enhancement training method according to claim 1, wherein the adjusting the human-computer interaction scheme according to the human-computer interaction scheme and a human-computer interaction evaluation result of the user, and pushing the adjusted human-computer interaction scheme to the user to perform a next human-computer interaction training until the purpose of cognitive enhancement of the user is achieved specifically comprises:
in the previous three human-computer interaction schemes, increasing the task level of the next human-computer interaction scheme by one level based on the task level of the current human-computer interaction scheme; and
at the beginning of the fourth human-computer interaction scheme, if the evaluation result of the current human-computer interaction scheme is higher than the evaluation result of the previous human-computer interaction scheme, increasing the task level of the next human-computer interaction scheme by one level, and if the evaluation result of the current human-computer interaction scheme is lower than the evaluation result of the previous human-computer interaction scheme, reducing the task level of the next human-computer interaction scheme by one level.

10. A cognitive enhancement training system based on neural regulation, comprising:
a data collection unit, connected with a central processing unit to obtain basic information of a user;
a rhythm frequency obtaining unit, connected with the central processing unit to obtain a rhythm frequency for the user to perform human-computer interaction;
a sensory stimulation mode obtaining unit, connected with the central processing unit to obtain a sensory stimulation mode for the user to perform human-computer interaction;
a human-computer interaction unit, connected with the central processing unit to obtain a human-computer interaction scheme and perform human-computer interaction with the user;
a human-computer interaction scheme evaluation unit, connected with the central processing unit to evaluate a human-computer interaction result of the user; and
a human-computer interaction scheme optimization unit, connected with the central processing unit and the human-computer interaction unit to adjust the human-computer interaction scheme based on an evaluation result of the human-computer interaction, and push the adjusted human-computer interaction scheme to the human-computer interaction unit;
wherein the central processing unit is configured to perform the cognitive enhancement training method according to claim 1.
